(19) United States
(12) Patent Application Publication
(10) Pub. No.: US 2006/0282151 A1
(43) Pub. Date: Dec. 14, 2006

(54) Medical device system

(76) Inventors: Jan Weber, Maple Grove, MN (US); Brian T. Berg, St. Paul, MN (US); Barry O'Brien, Barna (IE)

Correspondence Address: FISH & RICHARDSON PC, P.O. BOX 1022, MINNEAPOLIS, MN, 55440-1022, US

(21) Appl. No.: 11/151,977

(22) Filed: Jun. 14, 2005

(51) Int. Cl. A61F 2/06

(52) U.S. Cl. 623/1.11

(57) ABSTRACT

Methods and articles used to position a stent within a body are disclosed.

### TECHNICAL FIELD

This invention relates to medical device systems and related methods of using medical device systems.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprosthesis include stents and covered stents, sometimes called “stent-grafts”.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon expandable endoprosthesis. The balloon can be inflated to deform and to fix the expanded endoprosthesis at a predetermined position in contact with the lumen wall. The balloon can then be deflated, and the catheter withdrawn.

In another technique, a self-expandable endoprosthesis is formed of an elastic material that can be reversibly compacted and expanded, e.g., elastically or through a material phase transition. During introduction into the body, the endoprosthesis is restrained in a compacted condition on a catheter. Upon reaching the desired implantation site, the restraint is removed, for example, by retracting a restraining device such as an outer sheath, enabling the endoprosthesis to self-expand by its own internal elastic restoring force.

### SUMMARY

In an aspect, the invention features a medical device system including a catheter and a stent releasably attached to the catheter. The catheter of the medical device system has a catheter longitudinal axis and is selectively deflectable from the catheter longitudinal axis. The stent of the medical device system includes segments of different conductivity, which are arranged to define at least one closed, conductive loop that has a normal vector that is transverse to a stent longitudinal axis. The stent is releasably attached to the catheter so that the normal vector of the at least one closed, conductive loop is transverse to the catheter longitudinal axis.

In an aspect, the invention features a medical device system including a catheter being able to selectively bend towards a first direction; and a stent including a feature that determines a radial orientation of the stent, wherein the stent is releasably attached to the catheter.

In an aspect, the invention features a method of treating a body of a subject, including inserting into the body of the subject a stent that is releasably attached to a catheter, the stent including at least one closed conductive loop, the loop having a normal vector that is transverse to a stent longitudinal axis, the visibility of the stent varying under MRI as a function of the inproduct between the normal vector and the MRI B0 field axis and releasing the stent from the catheter at a release site to preferentially orient the stent to enhance visibility under MRI.

In an aspect, the invention features a method, including providing a stent having a conductive loop with a normal vector, the normal vector having a select orientation relative to the stent axis, deploying the stent at the target site such that the normal vector has a select orientation at the target site, and analyzing the target site by MRI.

Embodiments may include one or more of the following. The catheter is selectively deflectable in a first direction and the stent is attached to the catheter so that the normal vector substantially moves within the plane defined by the longitudinal axis and the first direction. The stent is attached to the catheter so that the normal vectors of all closed conductive loops are substantially parallel. The segments are arranged to define a single closed conductive loop having a normal vector that is transverse to the stent longitudinal axis. The catheter has a cross-sectional geometry that provides selective deflection. The cross-sectional geometry comprises an oval. The catheter further includes one or more stiffeners arranged to provide selective deflection. The catheter includes a polymer body. The catheter includes an electroactive polymer arranged to provide selective deflection. The stent is balloon expandable. The stent is self-expandable.

Embodiments may also include one or more of the following. The catheter is deflected towards the release site so that the normal vector of the loop is substantially parallel to the B0 field axis. The catheter is removed from the body of the subject. The release site is selected from the group consisting of thoracic aorta, renal artery, iliac artery, femoral artery, and subclavian artery. The stent is expanded at the release site. The stent is attached to the catheter so that during use the feature is positioned at a target site when the catheter is bent towards the first direction. The stent is attached to the catheter so that a normal vector defining the feature of the stent is substantially parallel and opposite to the first direction when the catheter is bent towards the first direction. The radial orientation feature is an opening within a wall of the stent. The feature is a closed, conductive loop.

Embodiments may also include one or more of the following. At the target site, the normal vector is substantially parallel to and in the direction of the main magnetic field applied during MRI imaging. The main magnetic field is substantially parallel to the feet-head axis of the body. The normal vector is transverse to the stent axis. The normal vector is oriented 25° to 90° relative to the stent axis. The stent has multiple loops, each of which is oriented transverse to the stent axis. All of the multiple loops have a common orientation angle relative to the stent axis. The stent has a single loop. The target site is in the renal arteries, iliac arteries, thoracic aorta, subclavian or femoral arteries. The orientation of an applied magnetic field is selected during MRI relative to the orientation of the normal vector.

Embodiments may have one or more of the following advantages. The stent can be releasably attached to the catheter so that the stent is delivered to a target site in a desired orientation. As a result, the stent can be positioned within the patient's body efficiently, which leads to improved health care. As a further result, in embodiments where the stent includes one or more conductive loops, the interior of the stent can be visualized with a magnetic resonance imaging (MRI) system because the conductive loops of the stent can be oriented to face the same direction as an incident magnetic field created by the MRI system.

Other aspects, features, and advantages will be apparent from the description and drawings, and from the claims.

### DESCRIPTION OF DRAWINGS

FIG. 1A is a perspective view of a stent.

FIG. 1B is an illustration of a circulatory system of a human body.

FIG. 1C is an enlarged view of a portion of the circulatory system labeled C in FIG. 1B with an embodiment of a medical device system including a stent releasably attached to a selectively deflectable catheter positioned within the portion.

FIG. 2 is a plan view of a portion of a stent wall.

FIG. 3A is a perspective view of a portion of the medical device system.

FIG. 3B is a cross-sectional view of the medical device system of FIG. 3A.

FIGS. 4A, 4B and 4C illustrate delivery of a medical device system.

FIG. 5 is a plan view of a portion of a stent wall.

FIG. 6 is a perspective illustration of an embodiment of a stent.

FIG. 7A is a perspective view of a bifurcated stent system.

FIG. 7B is a perspective view of a portion of the bifurcated stent system of FIG. 7A.

FIG. 8A is a cross-sectional view of an embodiment of a selectively deflectable catheter.

FIG. 8B is a cross-sectional view of another embodiment of a selectively deflectable catheter.

Like reference symbols in the various drawings indicate like elements.

### DETAILED DESCRIPTION

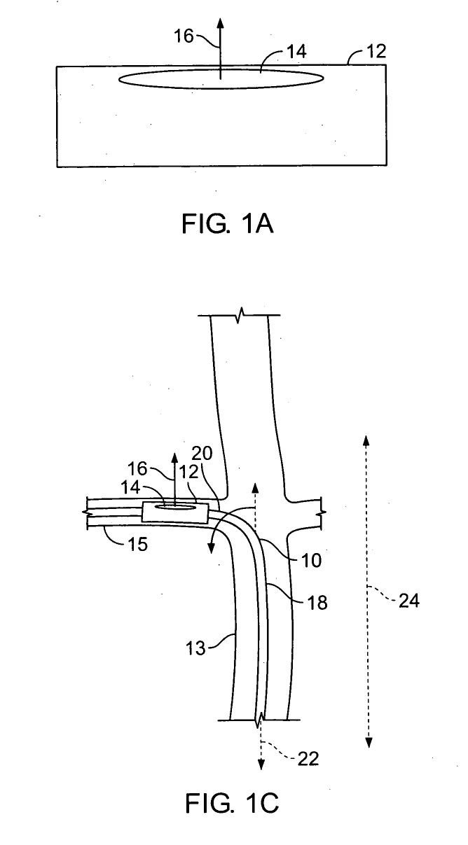

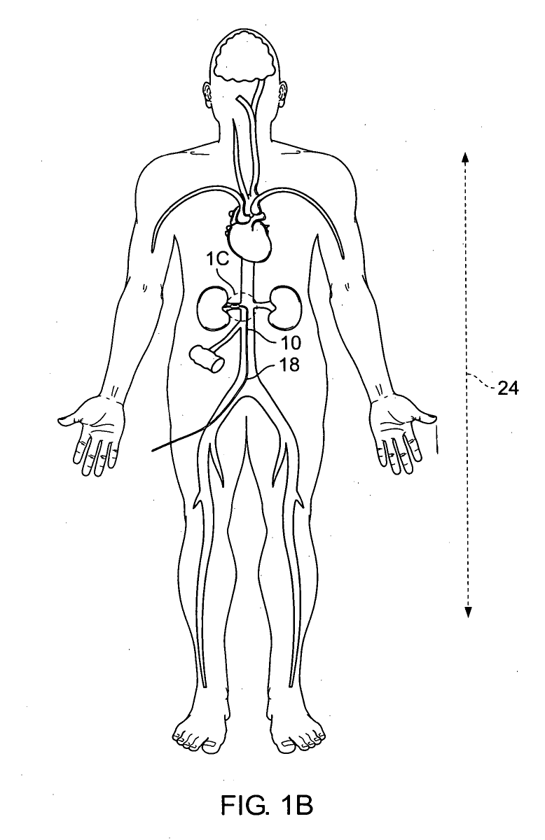

Referring to FIGS. 1A-1C, a medical device system 10 includes a stent 12 with a closed, conductive loop 14, having a normal vector 16, and a catheter 18 arranged to deliver the stent into a body lumen e.g. the renal arteries and deploy the stent at a target site such that the normal vector 16 has a select orientation that facilitates MRI. Stent 12 is releasably attached to the catheter 18 over a portion 20 that is selectively deflectable from the catheter's longitudinal axis 22 so that prior to deflection of catheter 18, normal vector 16 is transverse to longitudinal axis 22. Referring particularly to FIG. 1B, the catheter is delivered through a body lumen into a main renal artery 13, where the catheter is deflected to direct the catheter into a side branch 15. In this condition, the normal vector 16 is oriented substantially parallel to an axis 24 which generally corresponds to the head-feet direction of the patient.

Referring particularly to FIG. 1C, once positioned and implanted at a desired site (e.g., renal artery), stent 12 can be visualized with the use of a magnetic resonance imaging (MRI) machine. The MRI machine applies a main incident field, B0, that is typically static, and an RF field that is generally perpendicular to the main field BO. In this example, the field B0 is parallel to axis 24 along the head-feet direction of the patent. With normal vector 16 of closed loop 14 oriented in substantially the same direction as the incident main magnetic field B0, that is applied by MRI machine, the interior of stent 12 can be visualized. Conductive loops with normal vectors not substantially aligned with the main magnetic field B0 might generate eddy currents that can interfere with MRI visualization as they are not perpendicular to the incident RF-field, the main cause for largely varying electromagnetic fields. The device system controls radial positioning of normal vector 16 during stent delivery and implantation within the patient's body.

During MRI, an incident electromagnetic field is applied to a patient's body and, as a result, is applied to the stent. The visibility of the stent can vary under MRI as a function of the in product (vector inner product) of the stent normal vector and the B0 field axis. The magnetic environment of the stent can be constant or variable, such as when the stent moves within the magnetic field (e.g., from patient breathing resulting in diaphragm motion and motion of the renal artery or from blood flow) or when the incident magnetic field is varied. When there is a change in the magnetic environment of the stent, which can act as a coil or a solenoid, an induced electromotive force (emf) is generated according to Faraday's Law. The induced emf in turn can produce an eddy current within a closed, conductive loop that induces a magnetic field that opposes the change in magnetic field. The induced magnetic field can interact with the incident magnetic field to reduce (e.g., distort) the visibility of material within the lumen of the stent. As a result, by controlling (e.g., reducing, eliminating, or selecting the orientation of) closed, conductive loops that induce magnetic fields which oppose the incident magnetic fields (e.g., closed, conductive loops that have normal vectors not substantially aligned with the incident magnetic field) visualization is improved. MRI is further discussed in The Basics of NMR, Joseph P. Hornak, Ph.D., Copyright 1997-99, J. P. Hornak.

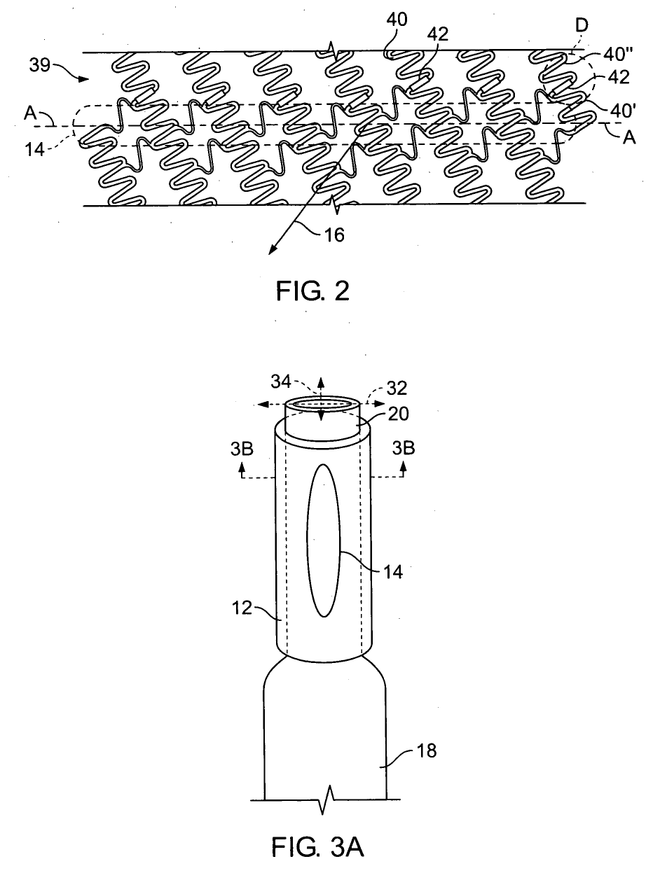

Referring to FIG. 2, a conductive loop or series of conductive loops having desirable orientations can be defined on a stent wall 39. (FIG. 2 illustrates a longitudinal portion of the wall of that stent extends along and around an axis A.) The stent wall 39 includes conductive segments 40 and current restrictive segments 42 that are arranged to define a conductive loop 14. Conductive segments 40 are formed from conductive materials, such as, for example, metals or metal alloys, that provide stent 12 with good mechanical properties (e.g., radial strength) to reinforce a passageway within the patient's body. Examples of conductive materials include stainless steel, titanium, niobium, tantalum and their alloys or shape memory alloys exhibiting temperature-induced superelastic behavior or pseudoelastic properties, e.g. Nitinol.

Segments 42 are formed from low or non-conductive materials such as, for example, ceramic and/or polymer materials, that are bonded to, inserted and/or embedded into the conductive material to increase resistance and thus prevent or reduce conduction through the segments 42, and thus reduce or eliminate the formation of conductive loops with non-selected orientations including, for example, around the circumference of stent 12. For example, the segments 42 can be formed by bonding a length of high resistance material to a lower resistance material making up segments 40. For example, segment 42 shown within section labeled D of FIG. 2 prevents current from flowing from conductive segment 40′ to conductive segment 40″. As a result of the pattern of segments, current within the wall of stent 12 will flow within closed, conductive loop 14 (defined by normal vector 16) as a result of changing electromagnetic fields, but not around the circumference of stent 12. The closed loop or loops are arranged such that the normal vector(s) can exhibit an orientation of a target site in a body lumen that facilitates viewing by MRI. Stents with desirable MRI conductive properties are described in Weber et al., U.S. Ser. No. 10/440,063, filed May 15, 2003. In embodiments, the stent can be reversibly compacted and expanded to provide radial self-expansion to stent 12. In other embodiments, the conductive segments are formed of a plastically deformable material for expansion by a balloon catheter. Examples of medical balloons are described in U.S. Pat. No. 5,195,969 to Wang and U.S. Pat. No. 5,270,086 to Hamlin, and hereby incorporated by reference. Stent materials and stent delivery are discussed in Heath U.S. Pat. No. 5,725,570.

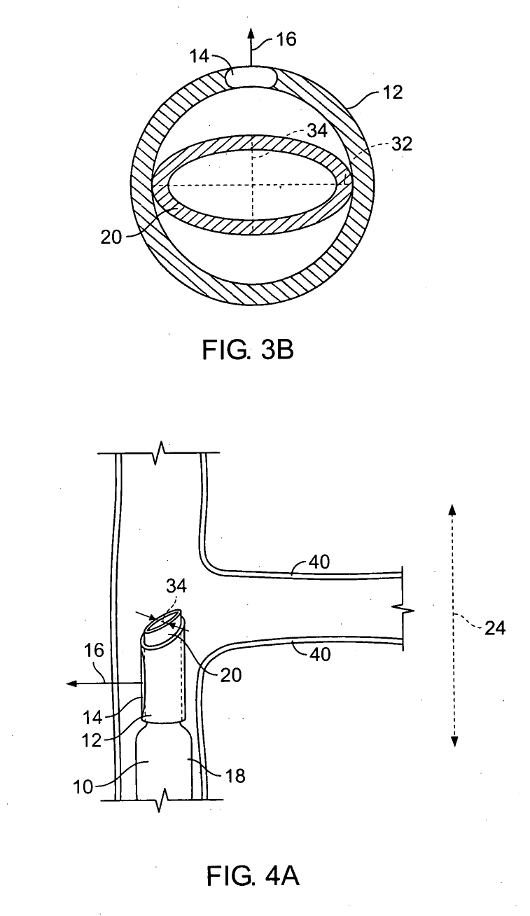

Referring to FIGS. 3A and 3B, in a particular delivery system, the catheter 18 has a selectively deflectable portion 20, with an elliptical (e.g. oval) cross-sectional geometry defined by a long axis 32 and a short axis 34. The mechanical properties (e.g., stiffness) of portion 20 along long axis 32 are different from the mechanical properties of portion 20 along short axis 34. Specifically, portion 20 has a greater stiffness along long axis 32 than along short axis 34 because of its cross-sectional geometry. As a result, when the catheter is delivered into a curved lumen, a compressive and/or transverse force is applied to longitudinal axis 22, and portion 20 bends in a direction parallel to short axis 34 rather than long axis 32. As a further result, when force is applied to catheter 18 to enter the renal artery (e.g., see FIGS. 1B and 1C), catheter 18 bends about 90° away from longitudinal axis 22 to enter renal artery and self-orients to have short axis 34 in the direction of curvature.

The position in which stent 12 is releasably attached to portion 20 is selected to provide desirable magnetic resonance imaging. For example, as shown in FIG. 3B, stent 12 is secured to portion 20 with normal vector 16 aligned with short axis 34. As a result, when catheter 18 bends to enter the renal artery, portion 20 self-orients so that the short axis 34 is in the direction of curvature and normal vector 16 parallel to longitudinal axis 24 (e.g., the direction of the incident magnetic field). As a result, visualizing the interior of stent 12 under MRI is facilitated because normal vector 16 is substantially aligned with the static magnetic field, thereby reducing the possibility of MRI interference caused by eddy currents. Portion 20 is sized to carry stent 12 through passageways in the patient's body. Materials used to form portion 20 are relatively strong so as to be able to support stent 12 and, at the same time, are relatively compliant so as to be able to navigate the natural contours of body passageways. Examples of suitable materials include polymers and metals. The catheter can be delivered over a guidewire. Alternatively or in addition, the catheter can include a pull wire attached to the distal end, which can be drawn from the proximal end to cause selective deflection.

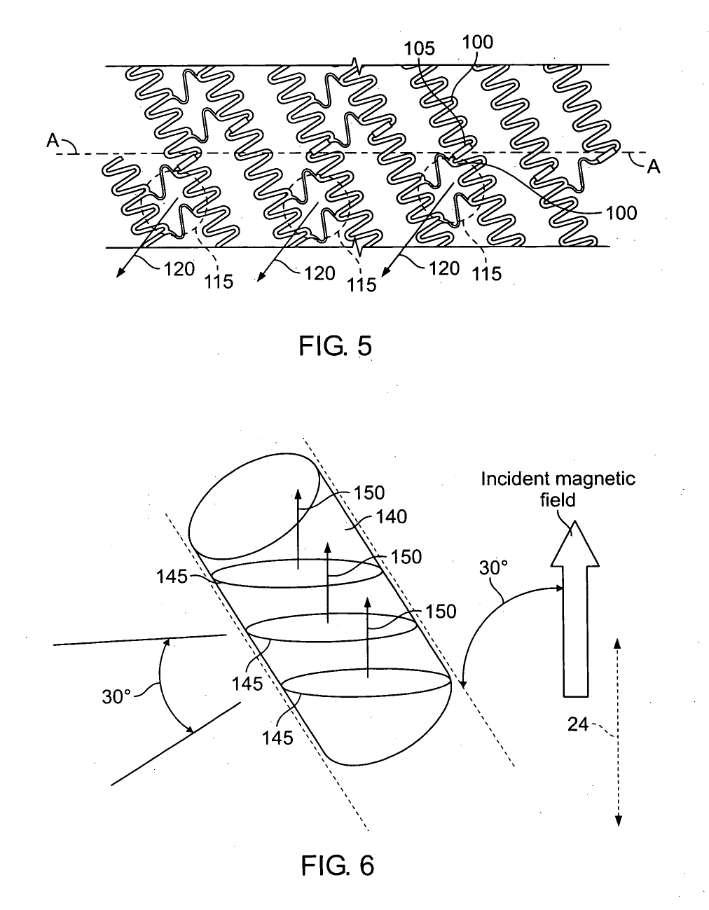

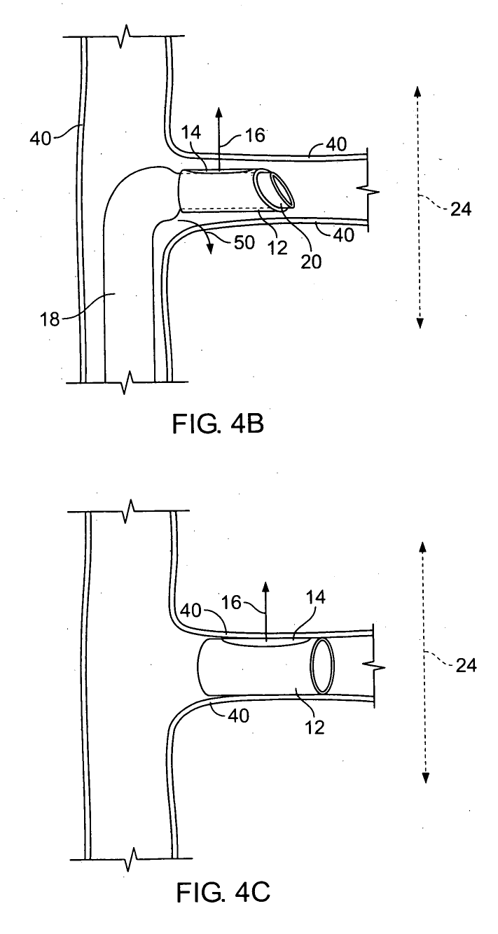

Referring to FIGS. 4A-4C, a method of using medical device system 10 is shown. Medical device system 10 is advanced through a patient's body towards the renal artery using conventional methods, such as by threading catheter 18 with stent 12 attached to portion 20 over an emplaced guide wire (not shown). For example, the catheter may be introduced into the femoral artery through an incision in the groin. Stent 12 is introduced and advanced in the patient's body in an unexpanded state so that catheter 18 can easily navigate through the patient's body without causing trauma to arterial walls 40. Referring particularly to FIG. 4B, because portion 20 of catheter 18 is selectively deflectable, catheter 18 self-orients so that portion 20 bends along short axis 34 in a preferred bending direction 50. As a result, portion 20 carries and positions stent 12 within the renal artery with normal vector 16 oriented parallel to longitudinal axis 24. Once located within renal artery (FIG. 4C), stent 12 is released from portion 20. The released stent expands to reinforce arterial walls 40. As expanded, stent 12 maintains its radial orientation (e.g., normal vector 16 remains parallel to longitudinal axis 24). Catheter 18 is then removed.

A medical professional at some later time can visualize the interior of stent 12 to monitor recovery (e.g., check to see if a stenosis has formed within the lumen of stent 12). Using an MRI system the medical professional can apply a static magnetic field aligned along the patient's longitudinal axis 24 to view the interior of stent 12 with little or no distortions generated by stent 12. In addition, the delivery and deployment can be monitored by MRI by visualizing changes in the MRI signal because stent orientation changes as it is delivered through the vasculature, creating variations in the signal.

Referring to FIG. 5, in embodiments, the stent can include more than a single closed conductive loop. The material forming the stent of the medical device system can include conductive and non-conductive segments 100 and 105, respectively, that form multiple (here, three) closed, conductive loops 115. Each of the multiple closed, conductive loops 115 is defined by a normal vector 120. Each of the normal vectors 120 has the same orientation and, as a result, can be aligned with the longitudinal axis of the body so as to reduce MRI distortions caused by eddy currents.

Referring to FIG. 6, the stent of the medical device system can include one or more defined conductive loops at selected desirable orientation(s). Stent 140 includes three conductive loops 145 that wrap around the circumference of the stent and have normal vectors oriented about 30° to the stent axis. (The loops are illustrated schematically as ellipses but could have other shapes, e.g., an undulating pattern.) For example, stent 140 can be positioned at a target site in a patient's iliac arteries which are at a thirty degree angle from longitudinal axis 24 of the patient's body and at a thirty degree angle from the applied incident magnetic field. As positioned within an iliac artery, conductive loops 145 do not create substantial eddy currents during an MRI scan because normal vectors 150, which define the conductive loops are aligned with longitudinal axis 24, perpendicular to the incident RF field. As a result, loops 145 do not interfere with the incident RF field and thus do not create MRI distortions. The example shown is in the right iliac; a stent with a mirror image loop pattern may be provided for use in the left iliac. The medical device system can be used to position and orient stents in other areas of the body. For example, the medical device system can be used to position stents in the thoracic aorta, subclavian arteries, and femoral arteries and bifurcations in the heart. Vector angles can vary between persons and can range between e.g., 20 degrees and 130 degrees. In embodiments, the stent can include multiple loops with different orientations. The different orientations can be used to control visualization of the stent in different lumens or portions of a lumen. The magnitude of the vectors can differ relative to one another. The stent can be placed at, e.g., venerable plague sites or sidebranches.

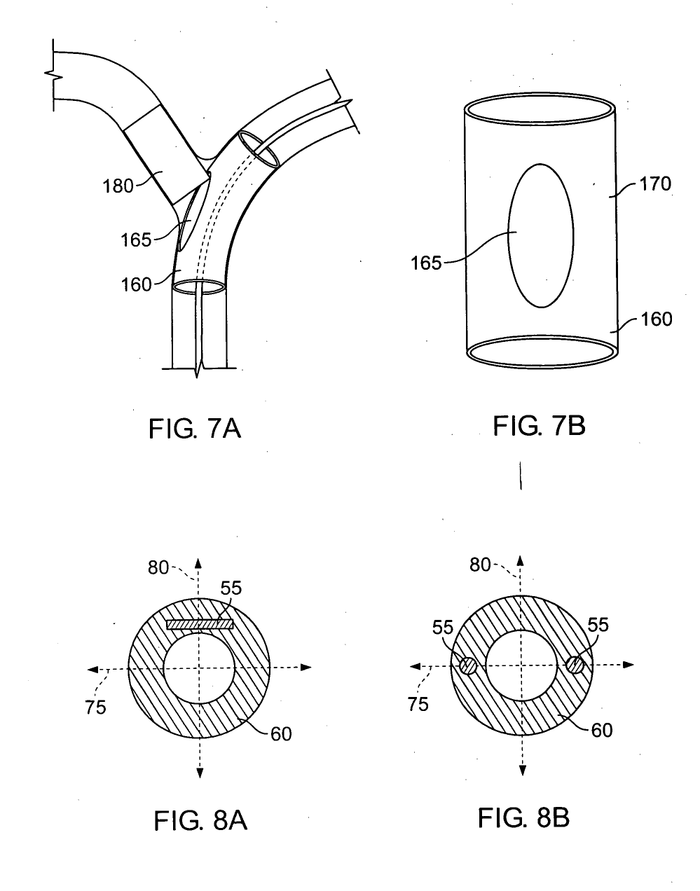

Referring to FIGS. 7A and 7B, a medical device system can be used to radially orient a stent 160 including an opening 165 within a stent wall 170. Using the medical device system described above, stent 160 can be positioned within a lumen and radially oriented so that a second stent 180 is positioned within opening 165 to form a bifurcated stent. The stents 160 and 180 can include different conductive loop orientations that correspond to the branching angle of the arterial portions into which the stent is deployed to facilitate visibility by MRI.

While certain embodiments have been disclosed, others are also possible. For example, although a portion of the catheter of the medical device system is described above as being selectively deflectable along a bending direction because of its oval cross-sectional shape, in certain embodiments, a portion of the catheter can be selectively deflectable and have a cross-sectional shape other than oval (e.g., rectangular, trapezoidal, square, circular). Selective deflection can be effected by use of a pull wire attached to a distal portion of the catheter and accessible from a proximal portion.

In embodiments, the catheter portion can be selectively deflectable along a bending direction because of the material(s) forming the portion. For example, the material(s) forming the portion can provide different material properties (e.g., stiffness) for different axes defining the portion's cross-sectional shape. In certain embodiments, the material(s) used to form portion 20 can include material(s) that change shape after a stimulus (e.g., electric energy, thermal energy) is applied. As a result of the material's change in shape, the cross-sectional shape of the catheter portion is altered to provide increased stiffness along one axis compared to another. Examples of suitable material(s) include shape memory alloys, such as titanium nickel alloys, and electroactive polymers, such as polyelectrolyte gels, ionic polymers, conducting polymers, and other electroactive polymers described in “Polymer Actuators” by Sommer-Larsen et al. (6.6 MB), Nordic Polymer DAYS, Stockholm, June 13-15, http://www.risoe.dk/fys-artmus/publications.htm, Proceedings of Actuator 2002, 8^{th} International Conference on New Actuators, Bremen, Germany, pp. 371-378.

In embodiments, the catheter portion can be selectively deflectable along a bending direction because of one or more stiffening elements disposed within a matrix material forming the portion. Referring to FIGS. 8A and 8B, stiffening elements 55 are positioned within a portion (e.g., portion 60 of FIG. 8A or portion 60′ of FIG. 8B) of a catheter to increase the stiffness along axis 75. As a result, the stiffness of portions 60 and 60′ are greater along axis 75 than along axis 80. Accordingly, portions 60 and 60′ are more likely to bend along axis 80 than along axis 75, and as a result, portions 60 and 60′ are selectively deflectable.

In embodiments, the orientation of the MRI field can be modified to a desired orientation relative to the orientation of the vector of the closed loop of the stent. In embodiments, the position of the patient in the MRI field can be modified to a desired orientation of the vector of the closed loop stent. For example, the arms could be positioned occurs the patient's abdomen to orient the radial and ulnar arteries perpendicular to the magnetic field.

All of the features disclosed herein may be combined in any combination. Each feature disclosed may be replaced by an alternative feature serving the same, equivalent, or similar purpose. Thus, unless expressly stated otherwise, each feature disclosed is only an example of a generic series of equivalent or similar features.

All publications, applications, and patents referred to in this application are herein incorporated by reference to the same extent as if each individual publication or patent was specifically and individually indicated to be incorporated by reference in their entirety.

Still other embodiments are in the following claims.

What is claimed is:

1. A medical device system comprising:
a catheter having a catheter longitudinal axis and being selectively deflectable from the catheter longitudinal axis; and
a stent including segments of different conductivity, the segments arranged to define at least one closed conductive loop, the loop having a normal vector that is transverse to a stent longitudinal axis,
wherein the stent is releasably attached to the catheter so that the normal vector of the at least one closed conductive loop is transverse to the catheter longitudinal axis.

2. The medical device system of claim 1, wherein the catheter is selectively deflectable in a first direction and the stent is attached to the catheter so that the normal vector substantially moves within a plane defined by the catheter longitudinal axis and the first diretion.

3. The medical device system of claim 1, wherein the stent is attached to the catheter so that the normal vectors of all closed conductive loops are substantially parallel.

4. The medical device system of claim 1, wherein the segments are arranged to define a single closed conductive loop having a normal vector that is transverse to the stent longitudinal axis.

5. The medical device system of claim 1, wherein the catheter has a cross-sectional geometry that provides selective deflection.

6. The medical device system of claim 5, wherein the cross-sectional geometry comprises an oval.

7. The medical device system of claim 1, wherein the catheter further includes one or more stiffeners arranged to provide selective deflection.

8. The medical device system of claim 7, wherein the catheter includes a polymer body.

9. The medical device system of claim 1, wherein the catheter includes an electroactive polymer arranged to provide selective deflection.

10. The medical device system of claim 1, wherein the stent is balloon expandable.

11. The medical device system of claim 1, wherein the stent is self-expandable.

12. A method of treating a body of a subject, the method comprising:
inserting into the body of the subject a stent that is releasably attached to a catheter, the stent including at least one closed conductive loop, the loop having a normal vector that is transverse to a stent longitudinal axis, the visibility of the stent varying under MRI as a function of the inproduct between the normal vector and the MRI BO field axis and
releasing the stent from the catheter at a release site to preferentially orient the stent to enhance visibility under MRI.

13. The method of claim 12 further comprising:
deflecting the catheter towards the release site so that the normal vector of the loop substantially parallel to the B0 field axis.

14. The method of claim 12 further comprising removing the catheter from the body of the subject.

15. The method of claim 12, wherein the release site is selected from the group consisting of thoracic aorta, renal artery, iliac artery, femoral artery, and subclavian artery.

16. The method of claim 12 further comprising:
expanding the stent at the release site.

17. A medical device system comprising:
a catheter being able to selectively bend towards a first direction; and
a stent including a feature that determines a radial orientation of the stent,
wherein the stent is releasably attached to the catheter.

18. The medical device system of claim 17, wherein the stent is attached to the catheter so that during use the feature is positioned at a target site when the catheter is bent towards the first direction.

19. The medical device system of claim 17, wherein the stent is attached to the catheter so that a normal vector defining the feature of the stent is substantially parallel and opposite to the first direction when the catheter is bent towards the first direction.

20. The medical device system of claim 17, wherein the catheter has a cross-sectional geometry that provides selective bending.

21. The medical device system of claim 20, wherein the cross-sectional geometry comprises an oval.

22. The medical device system of claim 17, wherein the catheter further includes one or more stiffeners arranged to provide selective bending.

23. The medical device system of claim 17, wherein the catheter includes an electroactive polymer arranged to provide selective deflection.

24. The medical device system of claim 17, wherein the feature is an opening within a wall of the stent.

25. The medical device system of claim 17, wherein the feature is a closed, conductive loop.

26. A method of treatment, comprising:
providing a stent having a conductive loop with a normal vector, the normal vector having a select orientation relative to the stent axis,
deploying the stent at the target site such that the normal vector has a select orientation at the target site, and
analyzing the target site by MRI.

27. The method of claim 26 wherein at the target site the normal vector is substantially parallel to and in the direction of the main magnetic field applied during MRI imaging.

28. The method of claim 27 wherein the main magnetic field is substantially parallel to the feet-head axis of the body.

29. The method of claim 27 wherein the normal vector is transverse to the stent axis.

30. The method of claim 29 wherein the normal vector is oriented 25° to 90° relative to the stent axis.

31. The method of claims 29 or 30 wherein the stent has multiple loops, each of which is oriented transverse to the stent axis.

32. The method of claim 31 wherein all of the multiple loops have a common orientation angle relative to the stent axis.

33. The method of claim 31 wherein the stent has a single loop.

34. The method of claim 26 wherein the target site is in the renal arteries, iliac arteries, thoracic aorta, subclavian or femoral arteries.

35. The method of claim 26 comprising
selecting the orientation of an applied magnetic field during MRI relative to the orientation of the normal vector.
